(19) United States
(12) Patent Application Publication
(10) Pub. No.: US 2011/0250641 A1
(43) Pub. Date: Oct. 13, 2011

(54) ZWITTERIONIC ACID-LABILE SURFACTANTS AND METHODS OF USE

(76) Inventors: Matthew Jacob Powell, Westover, WV (US); Trust Tariro Razunguzwa, Morgantown, WV (US); George Augustine O'Doherty, Boston, MA (US); Miaosheng Li, Morgantown, WV (US)

(21) Appl. No.: 13/081,640

(22) Filed: Apr. 7, 2011

Related U.S. Application Data
(60) Provisional application No. 61/322,059, filed on Apr. 8, 2010.

(51) Int. Cl. C12P 21/06; C25B 7/00; C07K 1/02; G01N 33/00; C07C 317/28; C07K 14/435

(52) U.S. Cl. 435/69.1; 564/292; 530/385; 436/86; 204/450; 204/456; 204/455; 204/548; 204/451

(57) ABSTRACT

A compound of the formula:

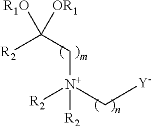

wherein R₁ is independently selected from C₁-C₁₀ alkyl or substituted alkyl, R₂ is independently selected from the group consisting of —H and C₁-C₆ alkyl or substituted alkyl, Y⁻ is an anion, m is an integer from 1 to 8 and n is an integer from 1 to 8 is described. Methods of making and using the compound are also described. The compound may comprise a zwitterionic acid-labile surfactant suitable for purification and identification techniques used in proteomics.

### CROSS-REFERENCE TO RELATED APPLICATION

This application claims the benefit of U.S. Provisional Application No. 61/322,059, filed on Apr. 8, 2010, which is incorporated herein by reference in its entirety.

### BACKGROUND

This application generally relates to zwitterionic acid-labile surfactants and methods of making and using the same.

Proteomics is the study of the structure and function of proteins and other molecules in biological systems. Some purification and identification techniques used in proteomics require the proteins and other molecules to be solubilized in an aqueous environment. Most proteins and other hydrophobic molecules or molecules with significant hydrophobic regions, however, are not readily soluble in an aqueous environment. Ionic and zwitterionic cleavable surfactants have been successfully used to facilitate the solubilization of proteins and other molecules in an aqueous environment.

Ionic and zwitterionic cleavable surfactants may generally comprise a polar (hydrophilic) group joined by a cleavable linker to a non-polar (hydrophobic) group. The ionic and zwitterionic cleavable surfactants may be cleaved by subjecting the surfactants to acidic conditions, basic conditions, photodegradation, thermal degradation, or chemical reduction. The cleavage by-products may be separated from the proteins or other molecules using standard isolation techniques. Conventional cleavable surfactants may generally comprise chemical structures that are complex to synthesize, require harsh conditions (e.g., pH 1-2) and/or long periods of time (up to several hours) to cleave, and/or generate cleavage by-products that interfere with purification and identification techniques.

Accordingly, more efficient zwitterionic cleavable surfactants and methods of making and using the same are desirable.

### SUMMARY

According to various embodiments, more efficient zwitterionic cleavable surfactants and methods of making and using the same are described.

In certain embodiments, zwitterionic acid-labile surfactants may generally comprise a compound of the formula:

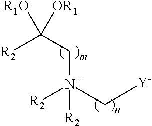

wherein each R₁ is independently selected from C₁-C₁₀ alkyl or substituted alkyl, each R₂ is independently selected from the group consisting of —H and C₁-C₆ alkyl or substituted alkyl, Y⁻ is an anion, m is an integer from 1 to 8 and n is an integer from 1 to 8.

In certain embodiments, methods of using a zwitterionic acid-labile surfactants may generally comprise mixing a solvent and a zwitterionic acid-labile surfactant of the formula:

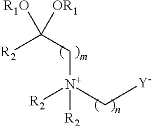

wherein R₁ is independently selected from C₁-C₁₀ alkyl or substituted alkyl, R₂ is independently selected from the group consisting of —H and C₁-C₆ alkyl or substituted alkyl, Y⁻ is an anion selected from the group consisting of —SO₃⁻, —PO₃⁻² and —PO₃H⁻, m is an integer from 1 to 8, and n is an integer from 1 to 8; contacting a sample and the mixture to generate a sample-surfactant complex; and cleaving the zwitterionic acid-labile surfactant to generate cleavage by-products

### DESCRIPTION OF THE DRAWINGS

The various embodiments described herein may be better understood by considering the following description in conjunction with the accompanying drawings.

FIG. 1 includes a chart illustrating various embodiments of zwitterionic acid-labile surfactants as described herein.

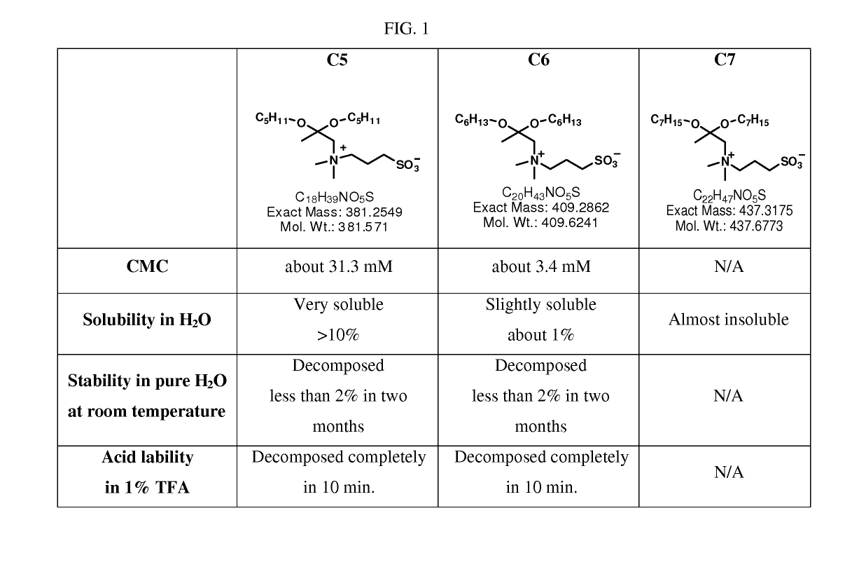

FIGS. 2A-2D includes mass spectra of certain embodiments of zwitterionic acid-labile surfactants as described herein and other surfactants.

### DESCRIPTION OF CERTAIN EMBODIMENTS

As generally used herein, the terms “consisting essentially of” and “consisting of” are embodied in the term “comprising”.

As generally used herein, the articles “a”, “an”, and “the” refer to “at least one” or “one or more” of what is claimed or described, unless otherwise indicated.

As generally used herein, the terms “include” and “have” mean “comprising”.

As generally used herein, the terms “about” and “approximately” refer to an acceptable degree of error for the quantity measured, given the nature or precision of the measurements. Typical exemplary degrees of error may be within 20%, 10%, or 5% of a given value or range of values.

All numerical quantities stated herein are approximate unless stated otherwise; meaning that the term “about” may be inferred when not expressly stated. The numerical quantities disclosed herein are to be understood as not being strictly limited to the exact numerical values recited. Instead, unless stated otherwise, each numerical value is intended to mean both the recited value and a functionally equivalent range surrounding that value. At the very least, and not as an attempt to limit the application of the doctrine of equivalents to the scope of the claims, each numerical parameter should at least be construed in light of the number of reported significant digits and by applying ordinary rounding techniques. Notwithstanding the approximations of numerical quantities stated herein, the numerical quantities described in specific examples of actual measured values are reported as precisely as possible.

All numerical ranges stated herein include all sub-ranges subsumed therein. For example, a range of “1 to 10” is intended to include all sub-ranges between and including the recited minimum value of 1 and the recited maximum value of 10. Any maximum numerical limitation recited herein is intended to include all lower numerical limitations. Any minimum numerical limitation recited herein is intended to include all higher numerical limitations.

As generally used herein, the terms “detergent” and “surfactant” refer to compounds and compositions that are amphiphilic, or possess both hydrophilic and hydrophobic properties and/or regions, and may facilitate the solubilization of proteins, other hydrophobic molecules, or molecules with significant hydrophobic regions in an aqueous environment.

As generally used herein, the term “cleave” refers to reducing or destroying the detergent properties of the surfactant. The term “cleave” may refer to separating the cleavable linker and the polar group and/or non-polar groups and/or degrading or disrupting the bond between the cleavable linker and the polar group and/or non-polar groups.

As generally used herein, the term “labile” refers to the property of a molecule or bond to undergo chemical, physical, or biological change, degradation, or disruption.

As generally used herein, the term “sample-surfactant complex” refers to the molecular complex that may be formed by certain embodiments of zwitterionic acid-labile surfactants and a sample.

As generally used herein, the term “sample” refers to any molecule that may be used with the zwitterionic acid-labile surfactants or methods described herein, such as hydrophobic molecules and molecules with significant hydrophobic regions, for example, but not limited to, proteins, peptides, polypeptides, polymers, nucleic acids, lipids, lipophilic cellular components, hydrophilic extracellular components, and any combinations thereof.

As generally used herein, when any variable occurs more than one time in a chemical formula, its definition on each occurrence is independent of its definition at every other occurrence.

As generally used herein, a dash (“-”) that is not between two letters or symbols is used to indicate a point of attachment for a substituent.

As generally used herein, the term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups, for example, methyl, ethyl, n-propyl, iso-propyl, n-butyl, sec-butyl, t-butyl, pentyl, n-hexyl, and cyclohexyl. In certain embodiments, a straight chain alkyl or branched chain alkyl may have 12 or fewer carbon atoms in its backbone (e.g., C₁-C₁₂ for straight chain, C₃-C₁₂ for branched chain), such as 10 or fewer, and such as 8 or fewer.

As generally used herein, the notations “a” and “b” in reference to an organic group, wherein a and b are each integers or an integer range, indicate that the group may contain a or b carbon atoms per group or that range of carbon atoms per group. The terminology “Cₐ-C_{b}” in reference to an organic group, wherein a and b are each integers, indicates that the group may contain from a carbon atoms to b carbon atoms per group.

Unless otherwise indicated, all compound or composition levels refer to the active portion of that compound or composition, and are exclusive of impurities, for example, residual solvents or by-products, which may be present in commercially available sources of any compounds or compositions.

All percentages and ratios are calculated based on the total weight of the compound or composition unless otherwise indicated.

In the following description, certain details are set forth in order to provide a better understanding of various embodiments of zwitterionic acid-labile surfactants and methods for making and using the same. However, one skilled in the art will understand that the embodiments described herein may be practiced without these details. In other instances, well-known structures and methods associated with zwitterionic acid-labile surfactants and methods of making and using the same may not be shown or described in detail to avoid unnecessarily obscuring descriptions of the embodiments of this disclosure.

This disclosure describes various features, aspects, and advantages of various embodiments of zwitterionic acid-labile surfactants and methods of use. It is understood, however, that this disclosure embraces numerous alternative embodiments that may be accomplished by combining any of the various features, aspects, and advantages of the various embodiments described herein in any combination or sub-combination that one of ordinary skill in the art may find useful.

In certain embodiments, zwitterionic acid-labile surfactants may generally comprise a non-polar (hydrophobic) group joined by a cleavable linker to a polar (hydrophilic) group. In certain embodiments, the zwitterionic acid-labile surfactants may comprise two shorter chain hydrophobic tails that individually bind weaker than conventional surfactants having the same net number of carbons in the hydrophobic region, but collectively bind nearly as well. The interaction between the cleavable linker and the polar group and/or non-polar group may be at least one of covalent bonding, ionic bonding, hydrogen bonding, and/or van der Waals bonding.

Without wishing to be bound to any particular theory, the zwitterionic acid-labile surfactant may be cleavable because the polar group may be separated from the non-polar group by disrupting the chemical bond between the cleavable linker and the polar and/or non-polar groups, or the polar and/or non-polar groups may be chemically modified such that the detergent properties of the surfactants are destroyed. Without wishing to be bound to any particular theory, the zwitterionic acid-labile surfactants may be labile because their surfactant properties may be eliminated by chemical processes other than bond cleavage. In certain embodiments, the zwitterionic acid-labile surfactant may be acid cleavable, i.e., acidic conditions may be used to cleave the bond between the cleavable linker and the polar group and/or non-polar groups. For example, the zwitterionic acid-labile surfactants described herein may be cleaved in 10 minutes at pH 2-3 using 1% trifluoroacetic acid (TFA).

In certain embodiments, the zwitterionic acid-labile surfactants may be hydrolyzed at a relatively low pH to generate cleavage by-products, including a zwitterionic, water-soluble or partially water-soluble compound (e.g., a polar head group) and a non-ionic, water-soluble or partially water-soluble compound (e.g., short to mid-length alcohols, such as pentanol, hexanol, heptanol, and octanol). The cleavage by-products may be removed from the sample-surfactant complex more readily than the original surfactants because they exhibit reduced, if any, detergent characteristics and/or do not readily bind to the sample. In at least one embodiment, the cleavage by-products may be washed away by solid phase extraction in which the sample may be bound to the surface of a reversed phase chromatographic bead.

In certain embodiments, the polar group and/or non-polar groups may improve the formation of a surfactant-sample complex. In at least one embodiment, the polar group and/or non-polar groups may improve the solubility of the cleavage by-products. The cleavage by-products may have reduced or negligible detergent characteristics. The cleavage by-products may be removed by standard isolation techniques. In certain embodiments, fewer by-products, such as adducts of the sample and non-degraded surfactant, may be formed. The cleavage by-products may be soluble in water. The cleavage by-products may have reduced or negligible interference with mass spectrometry analysis. The zwitterionic acid-labile surfactants may promote protein solubility and enzymatic digestion.

The zwitterionic acid-labile surfactants described herein may be especially suitable for purification and identification techniques in which conventional surfactants and/or cleavage by-products interfere with the purification and identification of the sample. The zwitterionic acid-labile surfactants may be suitable for solubilizing peptides, proteins, and other biomolecules, sample preparation and solid phase extraction, functioning in cell lysis protocols, protein extraction from cell lines, tissues, and biological samples, extraction of biomolecules from environmental samples, optimizing enzymatic digestions of proteins, isoelectric focusing, 2D gel separations, capillary electrophoresis, and electroelution buffers, and reducing surface adsorption losses due to non-specific interactions. Examples of proteomic purification and identification technologies that may benefit from the zwitterionic acid-labile surfactants described herein include, but are not limited to, ion-pair liquid chromatography, liquid chromatography, mass spectrometric detection, liquid-liquid extraction, solid phase extraction, cell lysis, and other technologies that may benefit from the removal of the surfactants after use. In at least one embodiment, the zwitterionic acid-labile surfactants may be suitable for isoelectric focusing during two-dimensional gel separations. The zwitterionic acid-labile surfactants may be a suitable replacement for conventional surfactants, such as CHAPS, NP-40, and the TRITON family of non-ionic detergents.

In certain embodiments, the zwitterionic acid-labile surfactants may generally comprise at least one non-polar group selected from the group consisting of hydrogen, C₁-C₁₀ alkyl or substituted alkyl; a polar group comprising an anionic group; and a cleavable linker comprising a ketal or an acetal.

In certain embodiments, the zwitterionic acid-cleavable surfactant may generally comprise a compound of Formula I:

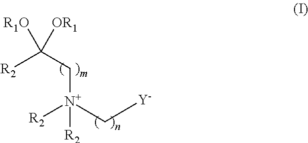

wherein R₁ is independently selected from C₁-C₁₀ alkyl or substituted alkyl, R₂ is independently selected from the group consisting of —H and C₁-C₆ alkyl or substituted alkyl, Y⁻ is an anion, m is an integer from 1 to 8 and n is an integer from 1 to 8. In certain embodiments, R₁ may be selected from —(CH₂)₀₋₉CH₃ alkyl, R₂ may be selected from —(CH₂)₀₋₅CH₃ alkyl, Y⁻ may be selected from the group consisting of —SO₃⁻, —PO₃⁻² and −PO₃H⁻, m may be an integer from 1 to 8 and n may be an integer from 1 to 8. In certain embodiments, Y may be selected from the group consisting of acetate, borate, carbonate, formate, phosphate, phosphonate, sulfate, and sulfonate ions. In at least one embodiment, Y may comprise a hard anionic charge selected from the group consisting of sulfate, sulfonate, phosphate, phosphonate and borate ions. In at least one embodiment, Y may comprise a weak anionic charge selected from the group consisting of carbonate, acetate, and formate ions.

In certain embodiments, R₁ may comprise a substituted C₁-C₁₀ alkyl selected from the group consisting of halogen substitution (e.g., —F, —Cl, —Br, or —I substitution), heterocyclic substitution, cyclic alkyl substitution, amide substitution, amine substitution, ester substitution, ether substitution, and phenyl substitution. The substituted C₁-C₁₀ alkyl may be at least one of fluoralkyl substitution, per-fluoroalkyl substitution, and benzene substitution.

In certain embodiments, R₂ may comprise a substituted C₁-C₆ alkyl selected from the group consisting of alkoxy substitution and halogen substitution (e.g., —F, —Cl, —Br, or —I substitution). The substituted C₁-C₆ alkyl may be at least one of fluoralkyl substitution and per-fluoroalkyl substitution.

In certain embodiments, zwitterionic acid-labile surfactants may generally comprise a compound represented by Formula II:

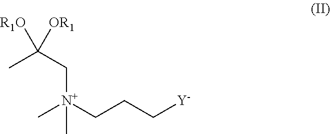

wherein R₁ is independently selected from C₁-C₁₀ alkyl or substituted alkyl, and Y⁻ is selected from the group consisting of —SO₃⁻, —PO₃⁻² and —PO₃H⁻.

In certain embodiments, zwitterionic acid-labile surfactants may generally comprise a compound represented by Formula III:

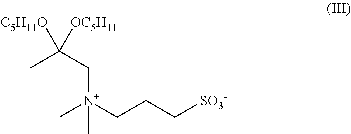

In at least one embodiment, the zwitterionic acid-labile surfactant may comprise 2,2-bis(pentyloxy)-N,N-dimethyl-N-(3-sulfopropyl)-1-propanaminium inner salt. The CMC may be about 31.3 mM. The zwitterionic acid-labile surfactants may be greater than 10% soluble in water. The zwitterionic acid-labile surfactants may decompose less than 2% in two months in pure water at room temperature. In at least one embodiment, the zwitterionic acid-labile surfactants may completely decompose in 10 minutes in 1% TFA.

In certain embodiments, zwitterionic acid-labile surfactants may generally comprise a compound represented by Formula IV:

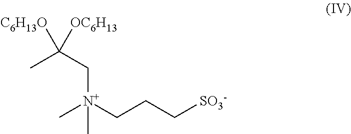

In at least one embodiment, the zwitterionic acid-labile surfactants may comprise 2,2-bis(hexyloxy)-N,N-dimethyl-N-(3-sulfopropyl)-1-propanaminium inner salt. The CMC may be about 3.4 mM. The zwitterionic acid-labile surfactants may be at least 1% soluble in water, such as 1-10%, and such as about 1%. The zwitterionic acid-labile surfactants may decompose less than 2% in two months in pure water at room temperature. In at least one embodiment, the zwitterionic acid-labile surfactants may completely decompose in 10 minutes in 1% TFA.

In certain embodiments, zwitterionic acid-labile surfactants may generally comprise a compound represented by Formula V:

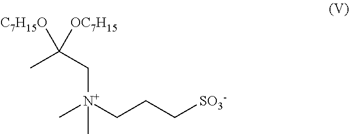

In at least one embodiment, the zwitterionic acid-labile surfactant may comprise 2,2-bis(heptyloxy)-N,N-dimethyl-N-(3-sulfopropyl)-1-propanaminium inner salt. The CMC may be less than 1 mM. The zwitterionic acid-labile surfactant may be substantially insoluble in water. The zwitterionic acid-labile surfactants may decompose less than 2% in two months in pure water at room temperature. In at least one embodiment, the zwitterionic acid-labile surfactants may completely decompose in 10 minutes in 1% TFA.

In certain embodiments, zwitterionic acid-labile surfactants may generally comprise a compound represented by any one of Formulas I-V having a rate of degradation of less than 30 minutes, such as from 4-24 minutes, and such as from 6-12 minutes. In at least one embodiment, the rate of degradation may be less than 10 minutes. In at least one embodiment, the rate of degradation may be 10 minutes. The rate of degradation generally relates to the rate of how easily the surfactant degrades. Without wishing to be bound to any particular theory, the rate of degradation may depend on the stability of the sample-surfactant complex. The stability of the sample-surfactant complex may depend on the chemical structure of the surfactant and/or the chemical structure of the sample-surfactant complex, e.g., electron donating groups and/or electron withdrawing groups. The rate of degradation may also depend on any contributions to increasing the kinetics of the degradation reaction, including, but not limited to, increasing the temperature and/or active mixing (e.g. sonicating and/or vortexing) of the sample-surfactant complex under acidic conditions.

In certain embodiments, a composition may comprise a sample-surfactant complex. The sample-surfactant complex may generally comprise a sample and a zwitterionic acid-labile surfactant according to any one of Formulas I-V. In certain embodiments, the composition may comprise a zwitterionic acid-labile surfactant according to any one of Formulas I-V and a protein mixture for electrophoresis. Without wishing to be bound to any particular theory, in solution, the hydrophobic tails of the surfactants may associate with the hydrophobic portion of the sample, e.g., proteins, via hydrophobic interactions. The hydrophilic heads of the surfactants may align outwardly from the hydrophobic tails to maximize the distance between the two opposing chemistries, and toward the bulk aqueous solvent where the hydrophilic heads may associate with the polar water molecules. The sample-surfactant complex may improve the solubility of the native (uncomplexed) sample because the hydrophilic heads may provide a cumulative improvement in the soluble nature of the sample-surfactant complex. In at least one embodiment, the solubility of the sample-complex may be greater than the solubility of the native sample. In at least one embodiment, the sample-surfactant complex may provide an increased potential for solvation and maintenance of a dissolved state.

The synthesis of the zwitterionic acid-labile surfactant compounds may be carried out using commercially available starting materials and synthetic methods described in Ono, D.; Yamamura, S.; Nakamura, M.; Takeda, T. J. Ole. Sci., 2004, 53 (2), 89-95 and Rouhana, L. L.; Jaber, J. A.; Schlenoff, J. B. Langmuir, 2007, 23(26), 12799-12801. The methods of synthesizing the zwitterionic acid-labile surfactants may produce isomers. Although the methods of using the zwitterionic acid-labile surfactants may not require separation of these isomers, such separation may be accomplished, if desired, by standard separation methods, such as, for example, preparative high performance liquid chromatography.

¹H NMR and ¹³C NMR spectra are recorded on a Varian 600 MHz spectrometer. Chemical shifts are reported relative to CDCl₃ (δ 7.24 ppm) or C₆D₆ (δ 7.16 ppm) for ¹H NMR and CDCl₃ (δ 77.0 ppm) or C₆D₆ (δ 128.4 ppm) for ¹³C NMR. Infrared (IR) spectra are obtained on a FT-IR spectrometer. Sorbtech 60A (230-400 mesh) silica gel is used for flash chromatography. Analytical thin-layer chromatography is performed with pre-coated glass-backed plates (K6F 60 Å, F254) and visualized by quenching of fluorescence and by charring after treatment with p-anisaldehyde or phosphomolybdic acid or potassium permanganate stain. R_{f} values are obtained by elution in the stated solvent ratios (v/v). Ether (Et₂O), methylene chloride (CH₂Cl₂) and toluene are dried by passing through an activated alumina (8×14 mesh) column with argon gas pressure. Commercial reagents are purchased from Fisher Scientific or Sigma-Aldrich and used without purification unless otherwise noted. Air and/or moisture-sensitive reactions are carried out under an atmosphere of argon/nitrogen using oven/flamed-dried glassware and standard syringe/septa techniques.

The following examples for the preparation of zwitterionic acid-labile surfactants are for illustrative purposes, and not intended to limit the scope of the zwitterionic acid-labile surfactants compounds and methods described herein. Additionally, in practicing the zwitterionic acid-labile surfactants and methods of making the same, one of ordinary skill in the art would understand that various modifications to the following procedures would be routine, in light of the teachings herein, and that such modifications would be within the spirit and scope of the zwitterionic acid-labile surfactants compounds and methods described herein.

To a solution of methyl pyruvate 1 (10.0 g, 98.0 mmol) in toluene (100 mL) was added 1-hexanol 2 (40.1 g, 392 mmol) and p-TsOH (186 mg, 0.98 mmol). The mixture was heated to reflux for 10 hours with azeotropic removal of water from the reaction mixture. The reaction was quenched with saturated NaHCO₃ (100 mL), and the reaction mixture was extracted with ethyl acetate (2×100 mL). The combined organic layers were washed with brine (100 mL) and dried over anhydrous sodium sulfate. The solvent was removed and the residue was purified by silica gel chromatography (1→10% ethyl acetate/hexane) to give hexyl 2,2-bis(hexyloxy)propanoate 3 (29.5 g, 84%) as a colorless oil: R_{f} (15% EtOAc/hexane)=0.53; IR (thin film, cm⁻¹) 2956, 2930, 2860, 1746 (C=O), 1467, 1280, 1137, 1062. ¹H NMR (600 MHz, CDCl₃) δ4.14 (t, J=7.2 Hz, 2H), 3.48 (ddd, J=9.0, 7.2, 6.6 Hz, 2H), 3.35 (ddd, J=9.0, 7.2, 6.6 Hz, 2H), 1.65-1.63 (m, 2H), 1.59-1.54 (m, 4H), 1.49 (s, 3H), 1.35-1.24 (m, 18H), 0.86 (t, J=7.2 Hz, 9H);¹³C NMR (150 MHz, CDCl₃) δ 170.2, 99.5, 65.4, 62.6, 31.7, 31.3, 29.7, 28.5, 25.8, 25.5, 22.6, 22.5, 21.9, 14.0, 13.9.

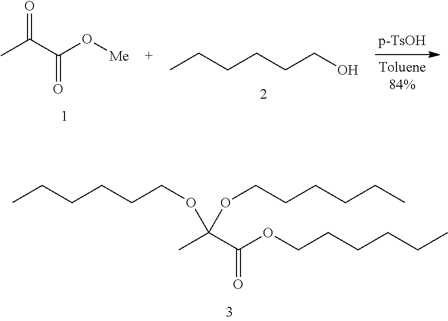

To a solution of ester 3 (35.8 g, 100.0 mmol) in THF (100 mL) was added a solution of dimethyl amine in THF (100 mL, 2M, 200 mmol). The solution was refluxed for 24 h in a pressure tube. The solvent was removed and the residue purified by silica gel chromatography (20→40% ethyl acetate/hexane) to give 2,2-bis(hexyloxy)-N,N-dimethylpropanamide 4 (27.7 g, 92%) as a colorless oil: R_{f} (50% EtOAc/hexane)=0.56; IR (thin film, cm⁻¹) 2929, 2863, 1652, 1461, 1389, 1368, 1104, 1061, 954, 909; ¹H NMR (600 MHz, CDCl₃) δ 3.43(dt, J=9.0, 6.6 Hz, 2H), 3.36 (dt, J=9.0, 6.6 Hz, 2H), 3.18 (s, 3H), 2.91 (s, 3H), 1.56-1.51 (m, 4H), 1.48 (s, 3H), 1.33-1.22 (m, 12H), 0.84 (t, J=7.2 Hz, 6H); ¹³C NMR (150 MHz, CDCl₃) δ 169.5, 101.4, 62.4, 38.0, 37.1, 31.6, 29.8, 25.9, 22.5, 20.4, 13.9.

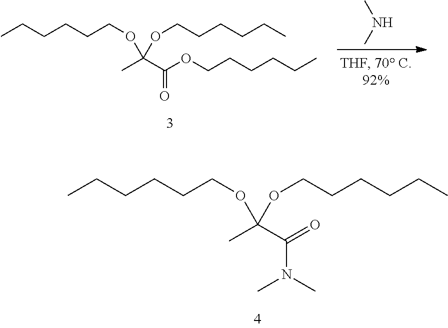

To a mixture of LiAlH₄ (3.46 g, 91.2 mmol) in Et₂O (200 mL) was added a solution of ester 3 (25.0 g, 82.9 mmol) in Et₂O (100 mL). After addition, the mixture was refluxed for 6 hours. The reaction mixture was cooled to 0° C. and quenched with ethyl acetate (20 mL) and H₂O (20 mL). The mixture was added saturated potassium sodium tartrate (300 mL) and stirred at 23° C. for 12 hours. The mixture was extracted with Et₂O (2×200 mL) and the combined organic layers were dried over anhydrous sodium sulfate. The solvent was removed and the residue was purified by silica gel chromatography (10→20% ethyl acetate/hexane +1% Et₃N) to give 2,2-bis(hexyloxy)-N,N-dimethylpropan-1-amine 5 (22.41 g, 94%) as a colorless oil: R_{f} (30% EtOAc/hexane+2% Et₃N)=0.62; IR (thin film, cm⁻¹) 2930, 2863, 2817, 2766, 1460, 1378, 1130, 1063, 1045, 955, 876; ¹H NMR (600 MHz, CDCl₃) δ 3.38 (t, J=7.2 Hz, 4H), 2.40 (s, 2H), 2.27 (s, 6H), 1.52-1.47 (m, 4H), 1.36 (s, 3H), 1.34-1.24 (m, 12H), 0.86 (t, J=7.2 Hz, 6H); ¹³C NMR (150 MHz, CDCl₃) δ 101.4, 64.4, 60.4, 47.2, 31.8, 30.1, 26.0, 22.6, 21.6, 14.0.

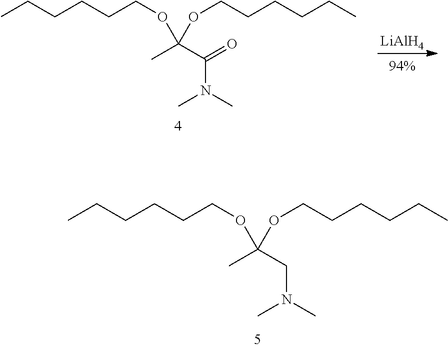

To a solution of amine 5 (5.80 g, 20.2 mmol) in acetone (40 mL) was added 1,3-propane sultone (4.93 g, 40.4 mmol). The reaction mixture was stirred at 60° C. for 10 h and then the reaction was quenched with DMAP (2.50 g, 20.5 mmol). The reaction mixture was stirred for another 10 h before being poured into aqueous NaHCO₃ (sat., 100 mL). The solution was extracted with chloroform (3×100 mL). The combined organic layers was washed with brine and dried over anhydrous sodium sulfate. The solvent was removed to give the zwitterionic surfactant 6 (7.18 g, 87%) as a white solid: R_{f} (80% MeOH/EtOAc)=0.58; IR (thin film, cm⁻¹) 2935, 2861, 2236, 1471, 1197, 1039, 912, 731; 1H NMR (600 MHz, CDCl₃)δ 3.86-3.83 (m, 2H), 3.51-3.47 (m, 4H), 3.49 (s, 2H), 3.30 (s, 6H), 2.87 (t, J=6.6 Hz, 2H), 2.30-2.23 (m, 2H), 1.55-1.50 (m, 4H), 1.48 (s, 3H), 1.32-1.24 (m, 12H), 0.86 (t, J=7.2 Hz, 6H); ¹³C NMR (150 MHz, CDCl₃) δ 100.6, 67.9, 65.8, 62.9, 52.5, 47.7, 31.5, 29.8, 25.9, 22.5, 21.6, 19.7, 14.0.

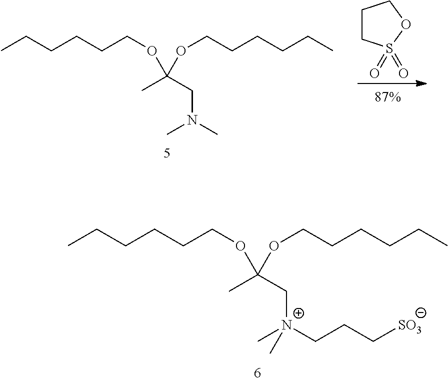

In certain embodiments, the zwitterionic acid-labile surfactants may facilitate the solubilization of proteins and other molecules in an aqueous environment. The zwitterionic acid-labile surfactants may be used in purification and identification technologies including, but not limited to, ion-pair liquid chromatography, liquid chromatography, such as high pressure liquid chromatography (HPLC), mass spectrometry (MS), such as electrospray ionization mass spectrometry (ESI) and matrix-assisted laser desorption/ionization mass spectrometry (MALDI-MS), liquid-liquid extraction, solid phase extraction, HPLC/MS, HPLC/UV analyses and other techniques that may benefit from the removal of the surfactant after use. The zwitterionic acid-labile surfactants may be used in other purification and identification technologies including, but not limited to, electrophoresis, capillary electrophoresis, electroelution, cell lysis and protein extraction from cell lines, tissues, and biological samples, selective protein extraction from biological samples, extraction of biomolecules from environmental samples, enzymatic digestion of proteins, disruption of protein-protein interactions, and protein denaturation.

In certain embodiments, a method of isolating a sample may generally comprise adjusting the sample to pH 6-12; mixing a solvent and a zwitterionic acid-labile surfactant according to any one of Formulas I-V; contacting the sample with the mixture to form a sample-surfactant complex; cleaving the surfactant from the sample-surfactant complex under acidic conditions to form cleavage by-products; and isolating the sample from the cleavage by-products. In certain embodiments, the method of isolating a sample may comprise agitating at least one of the sample, mixture, and sample-surfactant complex. Agitating may comprise sonication and/or vortexing. In at least one embodiment, the method of isolating a sample may comprise sonicating the sample-surfactant complex. In certain embodiments, the method may comprise performing mass spectrometry on the isolated sample. The cleavage by-products may be soluble in aqueous solvents, methanol, acetonitrile, ethanol, and isopropanol. The cleavage by-products may be soluble in at least one of the cleaved sample surfactant complex and isolated sample. In at least one embodiment, the cleavage by-products may comprises a sulfate salt.

In certain embodiments, adjusting the sample to pH 6-12 may comprise contacting the sample with an acid or a base. In at least one embodiment, adjusting the sample to pH 6-12 may comprise contacting the sample with an acid with the proviso that the acid is not a strong acid. In certain embodiments, adjusting the sample to pH 6-12 may comprise contacting the sample with a weak acid. The weak acid may be selected from the group consisting of formic acid, acetic acid, trifluoroacetic acid, heptafluorobutyric acid, citric acid, phosphoric acid, and boric acid. In certain embodiments, adjusting the sample to pH 6-12 may comprise contacting the sample with a strong base or weak base. The strong acid may be selected from the group consisting of hydrochloric acid, sulfuric acid, and nitric acid. The strong base may be selected from the group consisting of ammonium hydroxide, sodium hydroxide, and potassium hydroxide.

In certain embodiments, the solvent may be selected from the group consisting of water; 0-50% methanol; 0-50% acetonitrile; 5-500 mM ammonium bicarbonate buffer; 5-500 mM Tris-HCl buffer; 5-500 mM sodium phosphate buffer, 5-500 mM ammonium acetate buffer, and any combination thereof. In certain embodiments, the solvent may comprise any suitable solvent having a pH in the range of 7 to 10.

In certain embodiments, cleaving may comprise adjusting the sample-surfactant complex to pH 2-3. In certain embodiments, adjusting to pH 2-3 may comprise contacting the sample-surfactant complex with an acid. In at least one embodiment, adjusting to pH 2-3 may comprise contacting the sample-surfactant complex with a weak acid. The weak acid may be selected from the group consisting of formic acid, trifluoroacetic acid, heptafluorobutyric acid, and acetic acid. In at least one embodiment, adjusting to pH 2-3 may comprise contacting the sample-surfactant complex with a strong acid. The strong acid may be selected from the group consisting of hydrochloric acid, sulfuric acid, and nitric acid.

In certain embodiments, cleaving may comprise incubating the sample-surfactant complex. Cleaving may comprise incubating the sample-surfactant complex for less than four (4) hours at less than 99° C. In at least one embodiment, cleaving may comprise incubating the sample-surfactant complex from 5 minutes to 1 (one) hour at less than 50° C. In at least one embodiment, cleaving may comprise incubating the sample-surfactant complex from 5 minutes to 1 (one) hour at 4° C. to room temperature. In at least one embodiment, cleaving may comprise incubating the sample-surfactant complex for 10-30 minutes at 4-50° C. In at least one embodiment, cleaving may comprise incubating for 10 minutes at room temperature.

In certain embodiments, isolating the sample from the cleavage by-products may comprise performing purification and/or identification technologies. The purification and/or identification technologies may comprise any of the purification and/or identification technologies described herein. The purification and/or identification technologies may comprise at least one of reversed phase sample clean-up techniques and solid phase extraction techniques. The purification and/or identification technologies may be selected from the group consisting of ion exchange, hydrophilic interaction, reversed phase chromatographic preparations, and any combination thereof. The purification and/or identification technologies may comprise at least one of ultrafiltration and dialysis techniques.

In certain embodiments, the method of isolating a sample may comprise performing electrokinetic transport on at least one of the sample-surfactant complex and isolated sample. In at least one embodiment, performing electrokinetic transport may comprise electrophoresis. Electrophoresis may comprise isoelectric focusing, gel electrophoresis, free zone electrophoresis, and/or capillary electrophoresis. Gel electrophoresis may comprise polyacrylamide gel electrophoresis, including the tube, slab gel and capillary formats of polyacrylamide gel electrophoresis.

In certain embodiments the method of isolating a sample may comprise purifying the isolated sample. Purifying may comprise liquid-liquid extraction, solid-phase extraction, and liquid chromatography and other conventional separation methods.

In certain embodiments the method of isolating a sample may comprise performing enzymatic digestion of the sample-surfactant complex. Performing enzymatic digestion may comprise forming a sample-surfactant complex by contacting a sample and a zwitterionic acid-labile surfactant according to any one of Formulas I-V; incubating the sample-surfactant complex with a denaturing agent, such as dithiothreitol and beta-mercaptoethanol, for less than one (1) hour at less than 99° C., such 5 minutes to 1 (one) hour at 50-60° C., to reduce the cysteine-cysteine disulfide linkages; cooling the sample-surfactant complex mixture to less than 60° C., such as 20-40° C., and such as room temperature; incubating the sample-surfactant complex mixture with an alkylation agent, such as iodoacetamide, for less than one (1) hour at less than 99° C., such as 30 minutes at room temperature, in the dark, mostly dark, substantially dark, or completely dark; mixing the sample-surfactant complex with an enzyme; and incubating the mixture for less than 24 hours at less than 50° C., such as less than 12 hours at 20-40° C., and such as 4-8 hours at 37° C., with optional shaking. The alkylation agents, such as iodoacetamide, may be light-sensitive. The sample-surfactant complex mixture may be incubated with an alkylation agent in the dark, mostly dark, substantially dark, or completely dark to reduce the degradation of the alkylation agent.

In certain embodiments, performing enzymatic digestion may comprise forming a sample-surfactant complex by contacting a sample and a zwitterionic acid-labile surfactant according to any one of Formulas I-V (final concentration of 0.01-1.0%) in a buffered solution of 10-250 mM ammonium bicarbonate (pH 7.8); incubating the sample-surfactant complex with dithiothreitol (DTT) (5-500 mM) or beta-mercaptoethanol (5-500 mM) for less than one (1) hour, such as from 15 to 45 minutes at 50-60° C.; cooling the sample-surfactant complex mixture to 15 to 35° C., such as room temperature; incubating the sample-surfactant complex mixture with iodoacetamide (25-500 mM) for less than one (1) hour at less than 40° C. in the dark, such as for 15 to 45 minutes at room temperature; mixing the sample-surfactant complex with an enzyme; and incubating the mixture for less than 24 hours, such as 2 to 12 hours, at 20-40° C. with shaking.

In certain embodiments, performing enzymatic digestion may comprise forming a sample-surfactant complex by contacting a sample and a zwitterionic acid-labile surfactant according to any one of Formulas I-V (final concentration of 0.01-1.0%) in a buffered solution of 50 mM ammonium bicarbonate; incubating the sample-surfactant with 10 mM DTT for 30 minutes at 55° C.; cooling the sample-surfactant complex to room temperature; incubating the sample-surfactant complex mixture with 50 mM iodoacetamide for 30 minutes at room temperature in the dark; mixing the sample-surfactant complex with an enzyme; and incubating the mixture for 4-8 hours at 37° C. with shaking.

In certain embodiments, the enzyme may be selected from the group consisting of trypsin, Glu-C, Arg-C, Lys-C, Asp-N, chymotrypsin, and pepsin. The method may comprise contacting the sample-surfactant complex and trypsin in a ratio of 1:20 to 1:500 (enzyme:protein). The method may comprise adding 1 mM calcium chloride (CaCl₂) to the sample-surfactant complex and trypsin mixture. Proteolytically resistant proteins may require longer time periods and/or overnight digestion, such as from 2 hours to 24 hours, and such as at least 24 hours.

In certain embodiments the method of isolating a sample may comprise desalting the sample-surfactant complex with an enzyme. Desalting may comprise cleaving the sample-surfactant complex and/or degrading the surfactant of the sample-surfactant complex. In certain embodiments, desalting may comprise loading the mixture of the sample and degraded surfactant onto a solid phase extraction chromatographic media; washing away the salts and surfactant degradation products; and collecting the sample from the solid phase extraction media by elution. The solid phase extraction media may be selected from the group consisting of reversed phase chromatography, ion exchange chromatography, hydrophilic interaction liquid chromatography (HILIC), and any combination thereof. In certain embodiments, isolating the sample from the cleavage by-products may comprise at least one of ultrafiltration and dialysis techniques.

In certain embodiments, a method for analyzing a sample may generally comprise contacting a sample with a zwitterionic acid-labile surfactant according to any one of Formulas I-V to form a sample-surfactant complex and analyzing the sample-surfactant complex. Analyzing the sample-surfactant complex may comprise any of the purification and/or identification technologies described herein, such as, for example, but not limited to, electrophoresis, electroelution, high performance liquid chromatography, mass spectrometric detection, liquid-liquid extraction, solid phase extraction, and/or ion-pair liquid chromatography. The method for analyzing a sample may comprise degrading the surfactant. In at least one embodiment, the method for analyzing a sample may comprise purifying the sample after degrading the surfactant. In at least one embodiment, the method for analyzing a sample may comprise performing electrophoresis after degrading the surfactant.

In certain embodiments, a method for performing electrophoresis may generally comprise contacting a sample with a zwitterionic acid-labile surfactant according to any one of Formulas I-V to form a sample-surfactant complex; performing electrophoresis on the sample-surfactant complex; and degrading the surfactant after electrophoresis. Degrading the surfactant may comprise contacting the surfactant with an acidic solution. In certain embodiments, the method for performing electrophoresis may comprise purifying the sample after degrading the surfactant.

### EXAMPLES

The various embodiments of zwitterionic acid-labile surfactants and methods of use described herein may be better understood when read in conjunction with the following representative examples. The following examples are included for purposes of illustration and not limitation.

Comparisons of certain embodiments of zwitterionic acid-labile surfactants and commercially available cleavable surfactants are described. Cleavable surfactants generally exhibit various properties, including, but not limited to, lability (rate, efficiency, cleavage products), compatibility with mass spectrometry (level of signal suppression, adduct formation, artifact peaks, sample prep requirements) and other purification and identification technologies, such as polyacrylamide gel electrophoresis and mass spectrometry detection, and detergent strength and utility (critical micelle concentration (“CMC”), ability to perform electrophoresis).

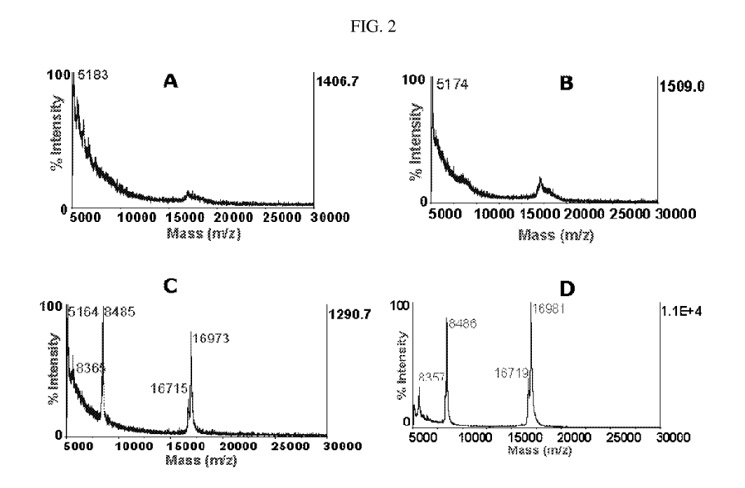

Referring to FIGS. 2A-2D, mass spectra of various embodiments of zwitterionic acid-labile surfactants and commercially available cleavable surfactants are illustrated. The mass spectra include 20 pmol myoglobin samples that were C8 spotted onto a MALDI plate pre-spotted with alpha-cyano-4-hydroxy-cinnamic acid (CHCA) MALDI matrix. FIG. 2A includes mass spectra of myoglobin with 0.1% CHAPS. FIG. 2B includes mass spectra of 0.1% CHAPS, acidified. FIG. 2C includes mass spectra of myoglobin with 0.1% zwitterionic acid-labile surfactants of Formula I. FIG. 2D includes myoglobin with 0.1% zwitterionic acid-labile surfactants of Formula I and 30 min TFA.

All documents cited herein are incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments of zwitterionic acid-labile surfactants and methods of making and using the same have been illustrated and described, it would be obvious to those skilled in the art that various other changes and modifications can be made without departing from the spirit and scope of the invention. Those skilled in the art will recognize, or be able to ascertain using no more than routine experimentation, numerous equivalents to the specific apparatuses and methods described herein, including alternatives, variants, additions, deletions, modifications and substitutions. This application including the appended claims is therefore intended to cover all such changes and modifications that are within the scope of this application.

What is claimed is:

1. A compound of the formula: 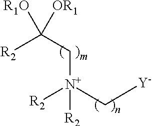
wherein R₁ is independently selected from C₁-C₁₀ alkyl or substituted alkyl, R₂ is independently selected from the group consisting of —H and C₁-C₆ alkyl or substituted alkyl, Y⁻ is an anion, m is an integer from 1 to 8 and n is an integer from 1 to 8.

2. The compound of claim 1, wherein R₁ is a substituted C₁-C₁₀ alkyl selected from the group consisting of halogen substitution, heterocyclic substitution, cyclic alkyl substitution, amide substitution, amine substitution, ester substitution, ether substitution, and phenyl substitution.

3. The compound of claim 2, wherein R₁ is a substituted C₁-C₁₀ alkyl selected from the group consisting of —F, —Cl, —Br, and —I substitution.

4. The compound of claim 2, wherein R₁ is a substituted C₁-C₁₀ alkyl selected from the group consisting of fluoralkyl substitution, per-fluoroalkyl substitution, and benzene substitution.

5. The compound of claim 1, wherein R₂ is a substituted C₁-C₆ alkyl selected from the group consisting of alkoxy substitution and halogen substitution.

6. The compound of claim 5, wherein R₂ is a substituted C₁-C₆ alkyl selected from the group consisting of —F, —Cl, —Br, and —I substitution.

7. The compound of claim 5, wherein R₂ is a substituted C₁-C₆ alkyl selected from the group consisting of fluoralkyl substitution and per-fluoroalkyl substitution.

8. The compound of claim 1, wherein R₁ is selected from —(CH₂)₀₋₉CH₃ alkyl, R₂ is selected from -(CH₂)₀₋₅CH₃ alkyl, Y⁻ is selected from the group consisting of —SO₃⁻, —PO₃⁻² and —PO₃H⁻, m is an integer from 1 to 8 and n is an integer from 1 to 8.

9. The compound of claim 1, wherein R₁ is selected from —(CH₂)₅₋₇CH₃ alkyl, R₂ is selected from —(CH₂)₀₋₂CH₃ alkyl, Y⁻ is selected from the group consisting of —SO₃⁻, —PO₃⁻² and —PO₃H⁻, m is an integer from 1 to 4 and n is an integer from 1 to 4.

10. The compound of claim 1, wherein R₁ is —(CH₂)₄CH₃, R₂ is —CH₃, and Y is —SO₃⁻, m is 1 and n is 3.

11. The compound of claim 1, wherein R₁ is —(CH₂)₅CH₃, R₂ is —CH₃, and Y is —SO₃⁻, m is 1 and n is 3.

12. The compound of claim 1, wherein R₁ is —(CH₂)₆CH₃, R₂ is —CH₃, and Y is —SO₃⁻, m is 1 and n is 3.

13. A method comprising:
mixing a solvent and a zwitterionic acid-labile surfactant of the formula: 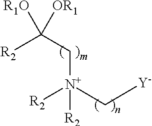
wherein R₁ is independently selected from C₁-C₁₀ alkyl or substituted alkyl, R₂ is independently selected from the group consisting of —H and C₁-C₆ alkyl or substituted alkyl, Y⁻ is an anion selected from the group consisting of —SO₃⁻, —PO₃⁻² and —PO₃H⁻, m is an integer from 1 to 8, and n is an integer from 1 to 8;
contacting a sample and the mixture to generate a sample-surfactant complex; and
cleaving the zwitterionic acid-labile surfactant to generate cleavage by-products.

14. The method of claim 13 comprising adjusting the sample to pH 6-12.

15. The method of claim 13 comprising isolating the sample from the cleavage by-products.

16. The method of claim 13, comprising performing mass spectrometry on the isolated sample.

17. The method of claim 13, wherein the cleaving comprises adjusting the sample-surfactant complex to pH 2-3 and incubating the sample-surfactant complex for 10-30 minutes at 4-50° C.

18. The method of claim 13, wherein the solvent has a pH in the range of 7 to 10.

19. The method of claim 13, comprising performing electrokinetic transport of the sample-surfactant complex.

20. The method of claim 13, comprising performing enzymatic digestion of the sample-surfactant complex.
